(19) United States
(12) Patent Application Publication
(10) Pub. No.: US 2018/0247718 A1
(43) Pub. Date: Aug. 30, 2018

(54) Method and apparatus to generate and provide relevant urgent/emergency care metrics to a user and automate several aspects of the provider process

(71) Applicants: Bhaskar Anepu, Allentown, PA (US); Ramanujam Lyengar, Holmdel, NJ (US); Shridar Lyengar, Morganville, NJ (US); Amit K Power, Reading, NJ (US)

(72) Inventors: Bhaskar Anepu, Allentown, PA (US); Ramanujam Lyengar, Holmdel, NJ (US); Shridar Lyengar, Morganville, NJ (US); Amit K Power, Reading, NJ (US)

(21) Appl. No.: 15/903,027

(22) Filed: Feb. 22, 2018

Related U.S. Application Data
(60) Provisional application No. 62/461,786, filed on Feb. 22, 2017.

(51) Int. Cl. G16H 80/00; G16H 10/60

(52) U.S. Cl. CPC G16H 80/00; G16H 40/20; G16H 10/60

(57) ABSTRACT

The present invention provides a novel way of assisting a patient in choosing the optimum emergency/urgent healthcare provider in an efficient way. It provides mechanisms to obtain critical pieces of information such as patient wait times, number of patients in a waiting room, patient symptom(s), patient's preferred health network, quality measures of a healthcare provider such a patient feedback, travel time from the patient location to the healthcare provider facility etc. and combine them to rank the healthcare providers.

### FIELD OF INVENTION

The present invention relates to a novel way of generating relevant data for a healthcare service provider and presenting this data in addition to other data to an end user to enable them to make a critical decision. More specifically, the invention relates to generating “data” pertinent to Emergency/Urgent/Immediate Care “providers” using wireless and/or wired sensor networks and/or proprietary “information” available from the Emergency/Urgent/Immediate Care “providers” and displaying this “information” in a “SW application” in a user friendly/readable format.

“provider” can be, but not limited to an Emergency Room in a hospital, an Urgent Care Center, a walk-in clinic or any other entity that provides emergency/urgent/immediate healthcare to patients

“data” can be, but not limited to, geographical location of the provider, historical average and/or real time average patient wait time at the provider facility, real time number of waiting patients at the provider facility, patient reviews and feedback of the provider, type of services provided etc.

“SW application” can be, but not limited to, web interface, mobile application, desktop application or any other user interface (text based or graphical) that can display/speak the “information” in a user readable/friendly format

“user” can be, but not limited to a person who is a patient seeking care at the provider or a person assisting a patient seeking care at the provider. Further in the document patient and user may be used interchangeably and it is understood that the relevant entity is to be considered according to the situation.

### BACKGROUND

Health care industry has seen exponential growth in recent years in technology applications to further enhance efficiencies on hospital front and user experience and convenience on the patient front. Still, there is a long way to go before this industry catches up with other industries like retail or banking in terms of technological embracement.

While most of the processes within a hospital and communication with the patients are being automated and made electronic, there are still scenarios where there is a gap that needs to be filled. The most striking example is Emergency/Urgent Care.

In the present day scenario, a person needing emergency/immediate care has very limited or even no means of making an informed decision to chose a provider that best suits their need at the desired time. In most cases the patient choses a known provider or the closest one to them at that point of time. This may not always be the best choice, especially considering the explosion in Urgent Care centers in the nation and their continuing growth. At the same time, the provider has absolutely no knowledge about a patient prior to their arrival at the provider facility. Bridging this gap can tremendously increase the chances of a patient getting the best care, in a timely manner, while helping the providers streamline their process and make it a lot more efficient.

Making use of the latest developments in technology, especially in the field of mobile communication and cloud based services, this gap can be filled and help both the patient and the provider in significant ways.

### BRIEF SUMMARY OF INVENTION

The present invention comprises a novel way of obtaining relevant provider specific data and presenting the same to an end user application securely to be used by a patient or someone responsible for the patient to make an informed decision to select the best care provider at any given point of time.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts an example where a wireless communications device carried by a patient entering the communication range of another wireless communications device located at a provider facility. This event is captured and securely communicated to a server.

FIG. 2 depicts an example where two patients each carrying a wireless communications device are located in the waiting area of a provider that is fitted with another wireless communications device that identifies the number of patients in the waiting area.

FIG. 3a and FIG. 3b depict an example illustrating a wireless communications device tracking the entry and exit of another wireless communications device carried by the patient and calculating the time spent by the patient in the waiting area.

FIG. 4 depicts an example where a patient carrying a wireless communications device approaches another wireless communications device located in the provider facility, which is used to obtain the patient's information and send it to an interface that can be accessed by the provider staff.

FIG. 5 depicts an example where the user selects “Preferred provider” as their preferred health network. When the user performs a search for emergency/urgent care providers using the SW application, preferred provider health network facilities are highlighted as stars on the map to differentiate them from other providers and are aggregated at the top of the list below the map.

### DETAILED DESCRIPTION OF THE INVENTION

The terminology used herein is for the purpose of describing particular embodiments only and is not intended to be limiting of the invention. As used herein, the term and/or includes any and all combinations of one or more of the associated listed items. As used herein, the singular forms “a”, “an” and “the” are intended to include the plural forms as well as the singular forms unless the context clearly indicates otherwise. It will be further understood that the terms “comprises” and/or “comprising”, when used in this specification, specify the presence of stated features, steps, operations, elements and/or components, but not preclude the presence or addition of one or more other features, steps, operations, elements, components and/or groups thereof.

Unless otherwise defined, all terms (including technical and scientific terms) used herein have the same meaning as commonly understood by one having ordinary skill in the art of which this invention belongs. It will be further understood that terms, such as those defined in commonly used dictionaries, should be interpreted as having a meaning that is consistent with their meaning in the context of relevant art and the present disclosure and will not be interpreted in an idealized or overly formal sense unless expressly so defined herein.

In describing the invention, it will be understood that a number of techniques and steps are disclosed. Each of these has individual benefits and each can also be used in conjunction with one or more and in cases all of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of invention and the claims.

The present disclosure is to be considered as an exemplification of the invention, and is not intended to limit the invention to the specific embodiments illustrated by the figures or description below.

Automated ways of detecting a patient when they arrive at a provider facility and check them in, obtaining patient wait times, number of patients in the waiting room at any given point of time and distribute this data to all relevant entities are discussed herein. A novel way of ranking providers with the help of afore mentioned metrics, when a user searches for a provider using the SW application is also presented. It is understood that all the communication between different entities involved, is to be done in a secure manner and the data stored on all of the entities is protected.

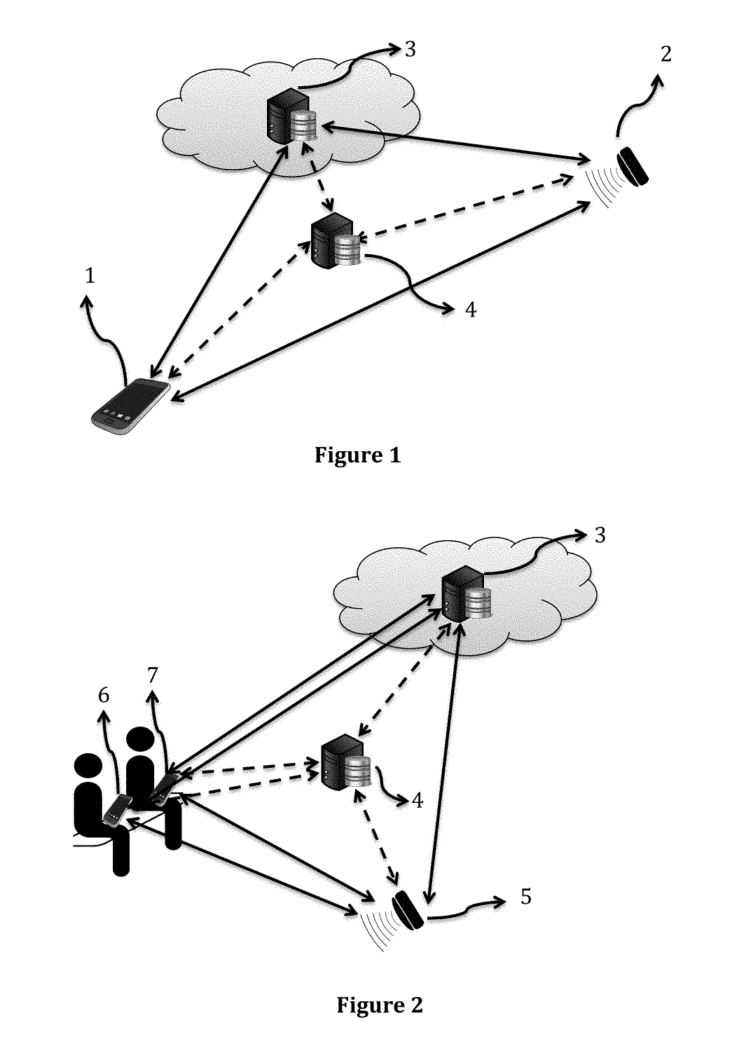

In preferred embodiment, FIG. 1 depicts a wireless transceiver 1 connected to another wireless transceiver 2 via a wireless link. Transceiver 1 may be a device being carried by the patient and transceiver 2 a fixed sensor at the provider facility. Transceivers 1 and 2 may be mobile user equipment such as a smartphone, a sensor device or any device that is capable of transmitting and/or receiving wireless communication signals such as but not limited to Bluetooth, Wi-Fi, RFID etc. As a patient carrying transceiver 1 comes within range of transceiver 2, transceiver 2 may start receiving a signal from transceiver 1 indicating its arrival. Alternatively, transceiver 2 may transmit a signal that transceiver 1 receives indicating that it entered the provider facility. In another embodiment, transceiver 1 and/or transceiver 2 may start sending the signals when they come in range of each other. This information may be uploaded to a remote server on the network cloud 3 directly or via a local server 4 so that this data can be accessed by different entities as needed. This process may be used for automatic check-in of the patient when they arrive at the provider facility.

In preferred embodiment, FIG. 2 depicts a waiting area at a provider facility where the patients wait before they are attended to, by the provider staff. Transceiver 5 is located in the waiting area that communicates wirelessly with transceivers 6 and 7 . . . that are carried individually by the different patients. Transceivers 5, 6 and 7 may be mobile user equipment such as the individual patient's smartphone, a sensor device or any device that is capable of transmitting and/or receiving wireless communication signals such as but not limited to Bluetooth, Wi-Fi, RFID etc. Transceivers 6 and 7 may be devices provided by the provider to the patients in order to track them or may be the same as transceiver 1 in FIG. 1 which the patients carried to the provider facility and was used for automatic check-in. Based on the communication between transceiver 5 and transceivers 6 and 7, transceiver 5 may calculate the number of patients present in the waiting area at any given point of time. This piece of information may be uploaded to a remote server on the cloud 3 directly or via a local server 4 for further use by other entities. Alternatively, transceiver 5 may upload the information pertinent to each of the transceivers 6 and 7 onto the remote server on cloud 3 directly or via the local server 4. This information on cloud 3 may then be used to calculate the number of patients in the waiting area at any given point of time. Alternatively, each of the transceivers 6 and 7 may indicate their presence in the waiting area by uploading relevant information about transceiver 5 to the remote server on cloud 3 directly or via the local server 4. This information on cloud 3 may then be used to calculate the number of patients in the waiting area at any given point of time. Any of these procedures may be used to provide the information about number of patients present in the waiting area at any given point of time. It is understood that, transceivers 6 and 7 are only used to illustrate a scenario with two patients in the waiting area and is not limited to this number in any way. This can be extended to any number of patients.

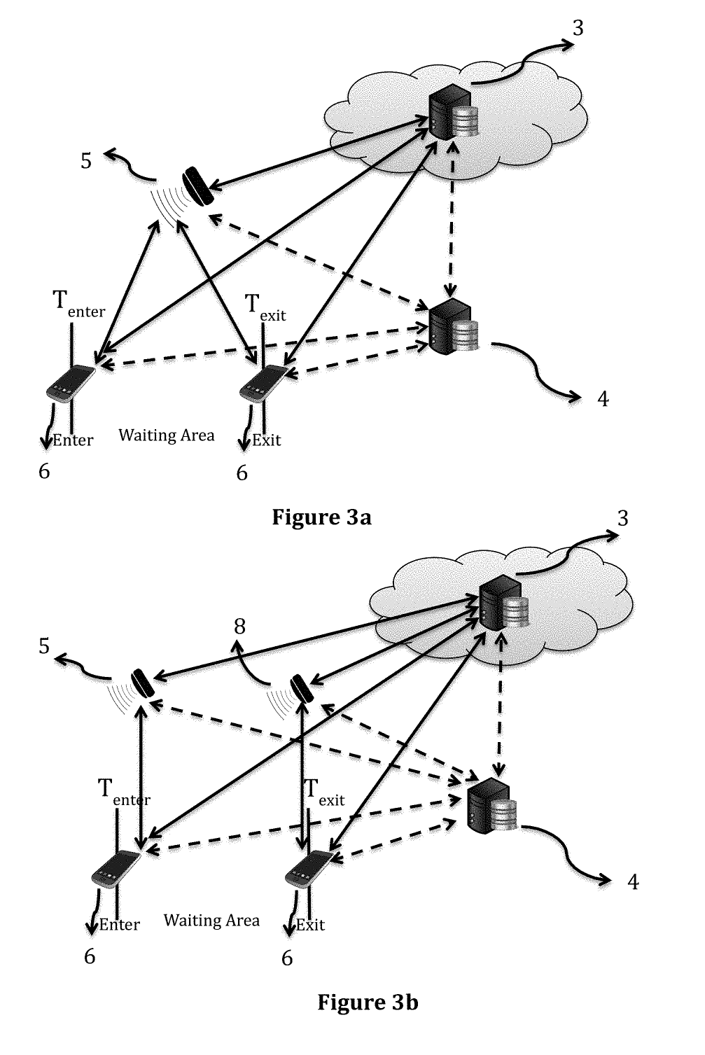

In preferred embodiment, FIG. 3a depicts transceiver 6 entering the waiting area at time Tₑₙₜₑᵣ and exiting the waiting area at time Tₑₓᵢₜ. These two times are communicated to the transceiver 5, which calculates the time spent by transceiver 6 in the waiting area as (Tₑₓᵢₜ-Tₑₙₜₑᵣ). This can be considered as a patient's wait time in the waiting area. This wait time may be uploaded to a remote server on the cloud 3 directly or via a local server 4. Alternatively, transceiver 5 may upload times Tₑₙₜₑᵣ and Tₑₓᵢₜ to the server and the patient's wait time, (Tₑₓᵢₜ-Tₑₙₜₑᵣ) may be calculated on the server. Alternatively, transceiver 6 may record Tₑₙₜₑᵣ and Tₑₓᵢₜ based on its communication with transceiver 5 and report the time (Tₑₓᵢₜ-Tₑₙₜₑᵣ) to the server or transceiver 6 may report times Tₑₙₜₑᵣ and Tₑₓᵢₜ to the server and the patient's wait time, (Tₑₓᵢₜ-Tₑₙₜₑᵣ) may be calculated on the server. In another embodiment as depicted in FIG. 3b, transceiver 5 may be used in obtaining the entry time, Tₑₙₜₑᵣ, of transceiver 6 and another transceiver 8 may be used to obtain the exit time of transceiver Tₑₓᵢₜ. These times may be utilized as explained above in determining the patient's wait time, (Tₑₓᵢₜ-Tₑₙₜₑᵣ). The average wait time of a patient may be determined by taking into consideration each patient's actual wait time and the number of patients over a predefined period of time.

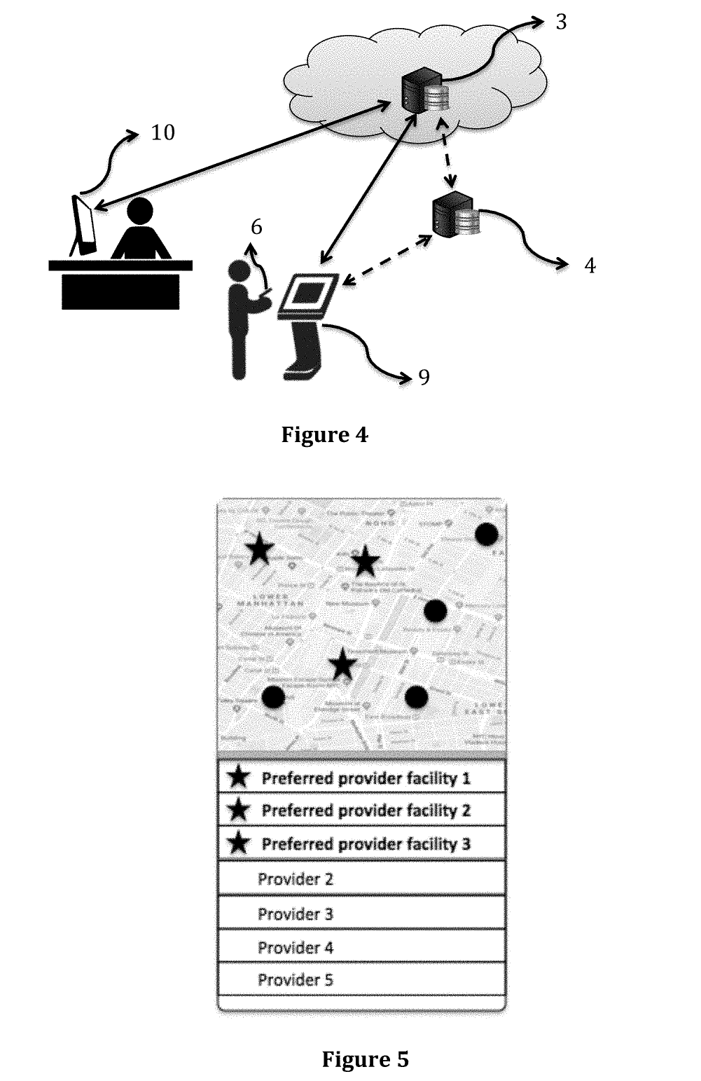

In preferred embodiment, FIG. 4 depicts transceiver 6 approaching device 9, which, for example, can be a touchscreen kiosk with wireless communications capabilities. At this time, device 9 may automatically detect the patient carrying transceiver 6 or the patient may manually enter their information into device 9. Device 9 then sends this information to the secure server, which pushes it to device 10, which, for example, can be a web interface being accessed via a web browser on the provider staff computer. The server can use the patient information in conjunction with other pieces of information such as the number of medical professionals at the provider facility, number of examining rooms, average time spent per patient etc. to compute average wait time at the facility.

In preferred embodiment, acuity level of the patient's illness may be determined based on information provided by the patient regarding the illness. This acuity level may be communicated to the provider before the patient's arrival at the provider facility enabling the provider to assign the patient to different queues and prepare the appropriate resources to handle the patient most efficiently. As an example, if the patient provides a low acuity level with symptoms pointing to an illness that can be handled by a nurse practitioner instead of a physician, the provider may direct the patient to a nurse practitioner as soon he/she arrives at the provider facility and hence avoid waiting for the physician. In another example, if the patient reports a high acuity level and the symptoms indicate resources such as a wheelchair or any other special assistance will be needed when the patient arrives; the provider may accommodate these ahead of time and redirect the patient accordingly when he/she arrives at the provider facility.

In preferred embodiment, when a user searches for a provider using the SW application, a list of providers within a pre-defined geographical area may be identified. These providers may be ranked based on the travel time to the provider from the location provided by the user, real time/average patient wait time at the provider facility, number of patients waiting at the provider facility, acuity level of the patient's illness and user reviews/feedback of the provider. Any or all these metrics may be obtained from the patient and/or from the public domain such as the Internet and/or from provider's database and/or from the mechanisms discussed in previous embodiments.

Provider Rank=F (Acuity Level, Travel Time, Wait Time, Number of Patients, Rating, Number of Reviews)

Any or all the above metrics may be considered in any possible combination.

In preferred embodiment, the user may select a preferred health network. When the user performs a search for a provider using the SW application, the facilities belonging to the selected preferred health network may be highlighted differently from the rest of the providers in the search results. For example, when the results are displayed on a map, the preferred health network facilities may be identified using different icons when compared to facilities not belonging to the preferred health network. In another example, the facilities belonging to the preferred health network may be identified using different colors than the facilities not belonging to the preferred health network. In another example, when the results are displayed as a list, all the facilities belonging to the preferred health network may be ranked higher and listed at the top before listing the facilities that do not belong to the preferred health network.

What is claimed is:

1. Mechanism to identify the number of patients waiting at a provider facility using each patient's smartphone or a wireless device that can communicate with another wireless device present at the providers facility

2. Mechanism to identify a patient's wait time at a provider facility by recording a patient's time of entry and time of exit to/from the waiting area, using the patient's smartphone or a wireless device that can communicate with another wireless device present at the providers facility.

3. Mechanism to identify patient's approximate wait time using patient's time of entry to provider facility, number of patients at the facility, number of clinical staff, number of examining rooms and the average time taken to treat each patient.

4. Calculation/equation/function/formula to rank different providers, taking into consideration any or all of the following metrics in any possible combination
a. Acuity level of the patient's illness
b. The travel time from the user provided start location to the providers facilities
c. Patient wait times at the providers facilities
d. Potential real time wait time before the patient is seen by clinical staff
e. Number of patients waiting at that time at the providers facilities
f. User ratings for the providers

5. Mechanism to let a patient select a preferred health network and highlight all the facilities belonging to the preferred network when the patient performs a search
a. When displayed on a map, differentiate them using a different icon or color from the facilities that do not belong to user selected preferred health network
b. When displayed as a list, display them at the top of the list before displaying facilities that do not belong to user selected preferred health network

6. The method to use and analyze data gathered from the sensors to predict patient flow volumes at different times during the day, the facilities from where patients come, other patient demographics to build a predictive model that will help in proper staffing, help in identifying facilities for potential other facilities, the kind of service needed to better triage etc
